(19) United States
(12) Patent Application Publication
(10) Pub. No.: US 2014/0073899 A1
(43) Pub. Date: Mar. 13, 2014

(54) PHOTOACOUSTIC SENSOR SYSTEM

(71) Applicants: Kristi Cohrs, Englewood, CO (US); Youzhi Li, Longmont, CO (US)

(72) Inventors: Kristi Cohrs, Englewood, CO (US); Youzhi Li, Longmont, CO (US)

(73) Assignee: Nellcor Puritan Bennett LLC., Boulder, CO (US)

(21) Appl. No.: 13/612,160

(22) Filed: Sep. 12, 2012

(51) Int. Cl. A61B 6/00

(52) U.S. Cl. 600/407

(57) ABSTRACT

A sensor fixture is provided for operatively attaching a photoacoustic (PA) sensor to a patient. The sensor fixture includes an acoustic coupling agent that is configured to allow the transmission of both acoustic energy and light therethrough. The sensor fixture includes a bracket configured to be affixed to skin of the patient. The bracket includes a cavity, a patient side, and a sensor side. The acoustic coupling agent is held within the cavity. The patient side includes a patient opening that is configured to expose the acoustic coupling agent along the patient side. The sensor side includes a sensor opening that is configured to expose the acoustic coupling agent along the sensor side. The sensor side includes a sensor cradle that is configured to hold the PA sensor such that the PA sensor is operatively attached to the acoustic coupling agent for receiving an acoustic response from the patient.

### FIELD

Embodiments of the present disclosure generally relate to medical devices, and more particularly to the use of photoacoustic sensors in patient monitoring.

### BACKGROUND

In the field of medicine, doctors often desire to monitor certain physiological characteristics of their patients. Accordingly, a wide variety of devices have been developed for monitoring many such characteristics of a patient. Such devices provide doctors and other healthcare personnel with the information they need to provide the best possible healthcare for patients. As a result, such monitoring devices have become an indispensable part of modern medicine. For example, clinicians may wish to monitor the patient's blood flow, cardiac output, and/or blood oxygen saturation, as such parameters may provide insight into the patient's respiratory and/or cardiac function. Deviation from normal or expected values may alert a clinician to the presence of a particular clinical condition.

Various techniques have been used to monitor physical characteristics of a patient such as blood flow, cardiac output, and/or blood oxygen saturation. But, some of such techniques may be undesirably invasive, for example, a specialized arterial catheter may be cannulated into the patient's arterial bloodstream. The invasiveness of such techniques may cause the patient discomfort, injury, and/or inconvenience.

### SUMMARY

Certain embodiments provide a sensor fixture for operatively attaching a photoacoustic (PA) sensor to a patient. The sensor fixture may include an acoustic coupling agent that is configured to allow the transmission of both acoustic energy and light therethrough. The sensor fixture may include a bracket configured to be affixed to skin of the patient. The bracket may include a cavity, a patient side, and a sensor side. The acoustic coupling agent may be held within the cavity. The patient side may face the skin of the patient when the bracket is affixed to the skin. The patient side of the bracket may include a patient opening that is configured to expose the acoustic coupling agent along the patient side. The sensor side of the bracket may include a sensor opening that is configured to expose the acoustic coupling agent along the sensor side. The sensor side of the bracket may include a sensor cradle that is configured to hold the PA sensor such that the PA sensor is operatively attached to the acoustic coupling agent for receiving an acoustic response from the patient.

The sensor fixture may include a sponge that is impregnated with the acoustic coupling agent. The sponge may be held within the cavity of the bracket and may be configured to allow the transmission of both the acoustic response and light therethrough.

The bracket may be configured to be affixed to the skin of the patient at a location that is adjacent an artery of the patient. The sensor cradle may be configured to hold the PA sensor such that an acoustic detector of the PA sensor is oriented approximately perpendicular to an artery of the patient.

The bracket may be configured to be affixed to the skin of the patient using an adhesive and/or a wrist band.

The sensor fixture may include a wrist band that is configured to be received around a wrist of the patient. The bracket may be mounted to the wrist band such that the wrist band is configured to affix the bracket to the skin of the patient adjacent a radial artery of the patient.

An adhesive may extend on the patient side of the bracket. The adhesive may be configured to affix the bracket to the skin of the patient.

The bracket may be configured to be affixed to the skin of the patient adjacent an ear of the patient.

The sensor cradle of the bracket may be configured to hold the PA sensor using a snap-fit connection, a press-fit connection, a slide tension connection, a threaded fastener, a latch, and/or a lock. The sensor cradle of the bracket may include a guide element that is configured to engage the PA sensor for orienting the PA sensor relative to the bracket.

The bracket may include an upper shell and a lower shell. The upper shell may include the sensor side of the bracket. The lower shell may include the patient side of the bracket. The upper shell and the lower shell may be connected together using a hinge, a living hinge, a clam shell arrangement, and/or a snap-fit connection.

The sensor fixture may be a disposable, single use, sensor fixture.

The sensor fixture may include a cover sheet configured to seal the patient opening when the sensor fixture is not being used and/or a cover sheet configured to seal the sensor opening when the sensor fixture is not being used.

Certain embodiments provide a PA sensor system that may include a PA sensor having a light source and an acoustic detector. The light source may be configured to emit light. The acoustic detector may be configured to receive an acoustic response from a patient. The PA sensor system may include a sensor fixture for operatively attaching the PA sensor to the patient. The sensor fixture may include an acoustic coupling agent that is configured to allow the transmission of both the acoustic response and light therethrough. The sensor fixture may include a bracket that is configured to be affixed to skin of the patient. The bracket may include a cavity, a patient side, and a sensor side. The acoustic coupling agent may be held within the cavity. The patient side may face the skin of the patient when the bracket is affixed to the skin. The patient side of the bracket may include a patient opening that is configured to expose the acoustic coupling agent along the patient side. The sensor side of the bracket may include a sensor opening that is configured to expose the acoustic coupling agent along the sensor side. The sensor side of the bracket may include a sensor cradle that is configured to hold the PA sensor such that the acoustic detector of the PA sensor is operatively attached to the acoustic coupling agent for receiving the acoustic response from the patient through the acoustic coupling agent.

Certain embodiments provide a method for measuring a physiological parameter of a patient using a PA sensor. The method may include affixing a sensor fixture to skin of the patient adjacent an artery of the patient such that an acoustic coupling agent of the sensor fixture engages the skin of the patient. The method may include mounting the PA sensor to the sensor fixture such that an acoustic detector of the PA sensor is operatively attached to the acoustic coupling agent for receiving an acoustic response from the artery of the patient through the acoustic coupling agent. The method may include transmitting light from the PA sensor, through the acoustic coupling agent, and into the artery of the patient to generate the acoustic response. The method may include receiving, at the acoustic detector, the acoustic response from the artery of the patient through the acoustic coupling agent.

Embodiments of the present disclosure may provide a sensor fixture that operatively attaches a PA sensor to a patient in a relatively quick and simple manner. The sensor fixture may enable the PA sensor to measure various physiological parameters of a patient by probing blood directly in a localized region of interest, such as, but not limited to, in a blood vessel.

Embodiments of the present disclosure may provide a sensor fixture that enables a PA sensor to measure various physiological parameters of a patient in a relatively non-invasive manner. Measurement of the physiological parameters using the sensor fixture may be less invasive than at least some known sensor systems.

Embodiments of the present disclosure may provide a disposable, single use, sensor fixture that enables a PA sensor to measure various physiological parameters of a patient.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

FIG. 1 is a block diagram illustrating a photoacoustic (PA) sensor system formed in accordance with an embodiment of the present disclosure.

FIG. 2 is a schematic illustration of an exemplary embodiment of a sensor fixture of the PA sensor system of FIG. 1.

FIG. 3 is a schematic illustration of another exemplary embodiment of a sensor fixture.

FIG. 4 is a schematic illustration of another exemplary embodiment of a sensor fixture.

FIG. 5 is a schematic illustration of the PA sensor system shown in FIG. 1 illustrating a PA sensor of the system operatively attached to a patient.

FIG. 6 is a flowchart illustrating an exemplary embodiment of a method for measuring one or more physiological parameters of a patient using the PA sensor system shown in FIGS. 1 and 5.

FIG. 7 is a perspective view of one specific exemplary embodiment of a PA sensor.

FIG. 8 is a perspective view of one specific exemplary embodiment of a sensor fixture.

FIG. 9 illustrates the PA sensor shown in FIG. 7 held by the sensor fixture shown in FIG. 8 at a variety of locations of a patient's body.

FIG. 10 is a perspective view of another specific exemplary embodiment of a sensor fixture.

FIG. 11 illustrates the PA sensor shown in FIG. 7 held by the sensor fixture shown in FIG. 10 at a patient's wrist.

FIG. 12 is a side elevational view of another specific exemplary embodiment of a PA sensor and another specific exemplary embodiment of a sensor fixture.

### DETAILED DESCRIPTION

One or more specific embodiments of the present disclosure will be described below. In an effort to provide a concise description of these embodiments, not all features of an actual implementation are described in the specification. It should be appreciated that in the development of any such actual implementation, as in any engineering and/or design project, numerous implementation-specific decisions must be made to achieve the specific goals of the developers, such as, but not limited to, compliance with system-related and/or business-related constraints, which may or may not vary from one implementation to another. Moreover, it should be appreciated that such a development effort might be relatively complex and/or relatively time consuming, but would nevertheless be a routine undertaking of design, fabrication, and/or manufacture for those of ordinary skill having the benefit of the present disclosure.

In certain medical contexts it may be desirable to ascertain various physiological parameters, such as, but not limited to, parameters related to individual blood vessels, parameters related to other discrete components of the vascular system, parameters that are not specific to individual or discrete blood vessels of the vascular system, parameters representative of a patient as a whole, and/or the like. Examples of such parameters may include, but are not limited to, oxygen saturation, hemoglobin count, perfusion, total hemoglobin (tHb) concentration, oxyhemoglobin saturation (SO₂), cardiac output (CO), and/or the like. One approach for measuring physiological parameters is referred to as photoacoustic (PA) spectroscopy.

PA spectroscopy utilizes light directed into a patient's tissue and/or blood to generate an acoustic response that may be detected and resolved to determine physiological information (i.e., parameters) of interest. Specifically, the light energy directed into the tissue and/or blood may be provided at particular wavelengths that correspond to the absorption profile of one or more blood and/or tissue constituents of interest. In some embodiments, the light is emitted as pulses (i.e., pulsed PA spectroscopy), though in other embodiments the light may be emitted in a continuous manner (i.e., continuous PA spectroscopy). The light absorbed by the constituent of interest results in a proportionate increase in the kinetic energy of the constituent (i.e., the constituent is heated), which results in the generation of pressure fluctuations that may be detected as an acoustic response (e.g., ultrasound). The acoustic response may be detected and used to determine the amount of light absorption, and thus the quantity of the constituent of interest, in the illuminated region. For example, the detected acoustic response may be proportional to the optical absorption coefficient of the blood and/or tissue constituent and the fluence of light at the wavelength of interest at the localized region being interrogated, e.g., a specific blood vessel. Furthermore, a phase difference between the detected acoustic response and the emitted light may indicated a position in the measurement (e.g., a depth in the tissue relative to the PA sensor).

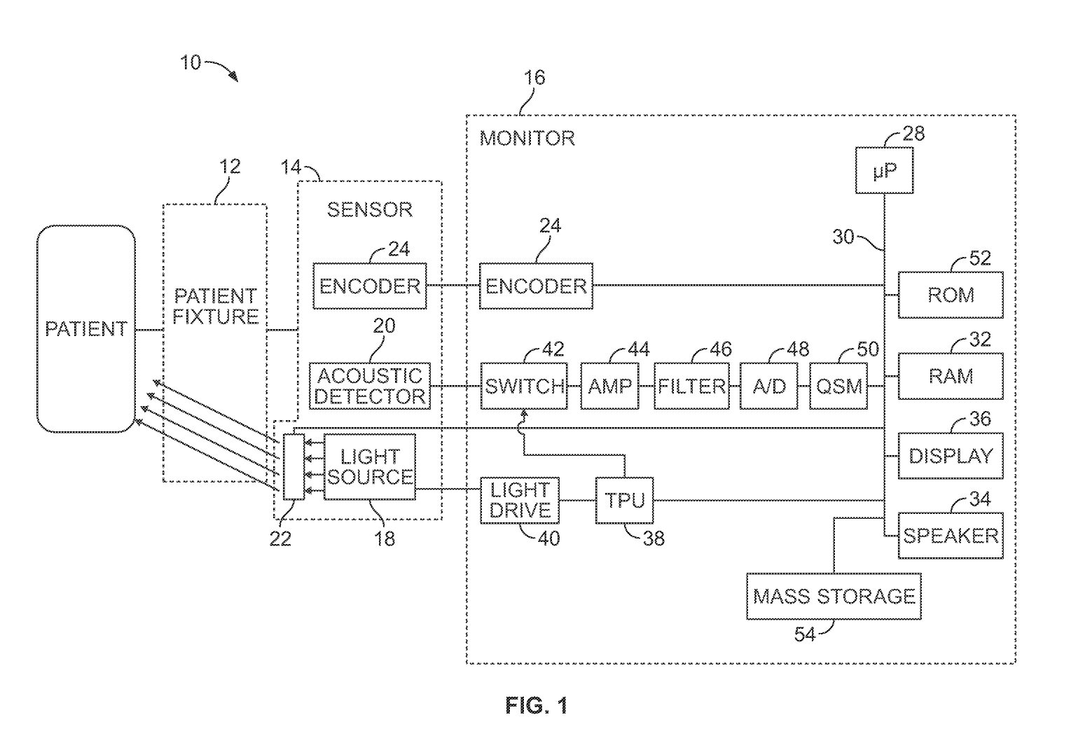

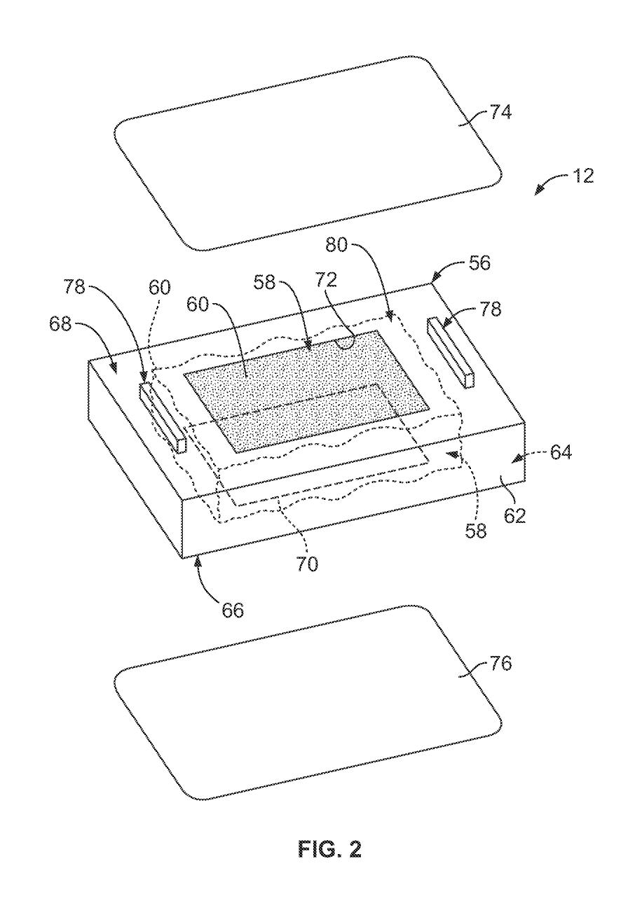

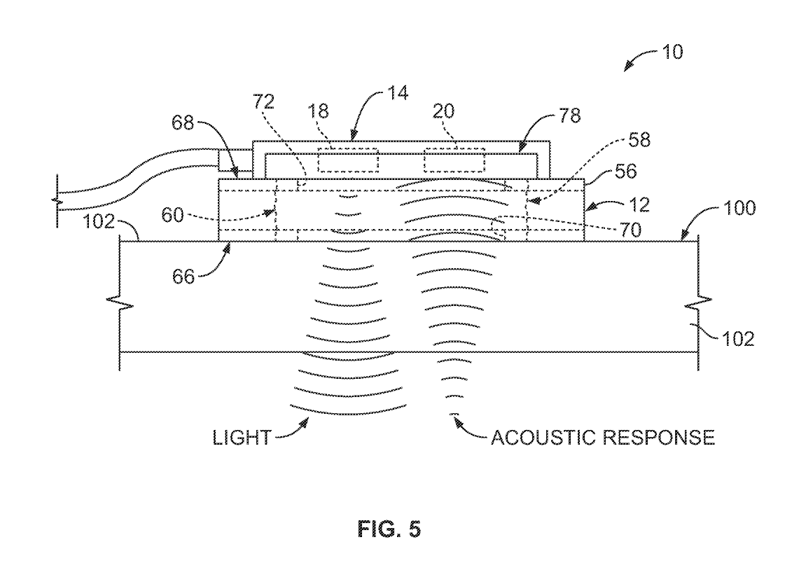

FIG. 1 is a block diagram of a PA sensor system 10 formed in accordance with an embodiment of the present disclosure. The system 10 includes a sensor fixture 12, a PA sensor 14, and a monitor 16. The sensor fixture 12 operatively attaches the PA sensor 14 to the patient. In other words, the sensor fixture 12 is used to mount the PA sensor 14 to the patient in operative connection with the patient's skin. The sensor fixture 12 will be described in more detail below with reference to FIGS. 2 and 5. The PA sensor 14 may emit spatially modulated light at certain wavelengths into a blood vessel (e.g., an artery) of the patient and may detect an acoustic response generated in response to the emitted light. The monitor 16 may be capable of calculating physiological parameters of the patient based on signals received from the PA sensor 14 that correspond to the detected acoustic response.

The PA sensor 14 includes one or more light sources 18 and one or more acoustic detectors 20. The PA sensor 14 may be used to directly or indirectly measure any physiological parameter of the patient, such as, but not limited to, the amount or concentration of a constituent of interest in a localized region (e.g., a blood vessel), oxygen saturation, hemoglobin count, perfusion, tHb concentration, SO₂, CO, and/or the like. In some embodiments, one or more of the physiological parameters directly measured by the PA sensor 14 is used to calculate a physiological parameter (e.g., oxygen saturation, hemoglobin count, perfusion, tHb concentration, SO₂, CO, and/or the like) of the patient. In other words, the PA sensor 14 may be used to indirectly measure the physiological parameter(s) of the patient. The light source 18 is configured to emit light and may be a pulsed light source that emits light in pulses and/or may be a continuous wave light source that emits light continuously. In some embodiments, the PA sensor 14 is configured to emit both pulsed light and continuous wave light.

The PA sensor 14 may include any number of the light sources 18. Each light source 18 may be any suitable type of light source. For example, in some embodiments, the light source 18 may include one, two, or more light emitting components (such as, but not limited to, lasers, light emitting diodes (LEDs), and/or the like) adapted to transmit light at one or more specified wavelengths. In some embodiments, the light source 18 may include a laser diode and/or a vertical cavity surface emitting laser (VCSEL). The laser diode may be a tunable laser, such that a single diode may be tuned to various wavelengths corresponding to a number of different absorbers of interest in the tissue and/or blood. Depending on the particular arrangement of the PA sensor 14, the light source 18 may be associated with one or more optical fibers for transmitting the emitted light into the blood and/or tissue.

The light emitted by the light source 18 may be any suitable wavelength or wavelengths (such as, but not limited to, a wavelength between approximately 500 nm and approximately 1000 nm, a wavelength between approximately 600 nm and approximately 900 nm, and/or the like) that is absorbed by a constituent of interest in the blood and/or tissue. For example, wavelengths between about 500 nm to about 600 nm, corresponding with green visible light, may be absorbed by deoxyhemoglobin and oxyhemoglobin. Additionally or alternatively, and for example, red wavelengths (e.g., about 600 nm to about 700 nm), infrared wavelengths, and/or near infrared wavelengths (e.g., about 800 nm to about 1000 nm) may be used.

The light emitted by the light source 18 may be intensity modulated. The light emitted by the light source 18 may be intensity modulated at any suitable frequency, such as, but not limited to, between approximately 0.5 MHz and approximately 10.0 MHz, greater than approximately 10.0 MHz, and/or the like.

The light emitted by the light source 18 may be spatially modulated. For example, in some embodiments, the PA sensor 14 includes a spatial modulator 22, which may be any suitable type of spatial modulator, such as, but not limited to, a Holoeye® LC-R 2500 liquid crystal spatial light modulator. In some embodiments, the modulator 22 may be associated with additional optical components (such as, but not limited to, lenses, reflectors, refraction gradients, polarizers, and/or the like) through which the spatially modulated light passes before reaching the blood and/or tissue of the patient.

The PA sensor 14 may include any number of the acoustic detectors 20. Each acoustic detector 20 may be any suitable type of acoustic detector suitable for receiving an acoustic response generated by the blood and/or tissue when exposed to the emitted light. In some embodiments, the acoustic response may be a pressure fluctuation, an acoustic shock wave, a thermal wave, and/or any other non-optical wave generated by the conversion of absorbed light energy into kinetic energy. The acoustic detector 20 may be suitable for measuring the frequency and/or amplitude of the acoustic response, the shape of the acoustic response, and/or the time delay associated with the acoustic response with respect to the light emission that generated the acoustic response. While a pulsed light PA sensor may utilize a comparably more complex acoustic detector suitable for detecting the acoustic response generated in response to relatively higher-powered pulsed light, an acoustic detector for a continuous wave PA sensor may be a standard detector model suitable for detecting acoustic responses generated using relatively lower power light emissions.

In some embodiments, the acoustic detector 20 may be one or more ultrasound transducers (such as, but not limited to, a piezo composite transducer, a PVDF transducer, and/or the like) suitable for detecting ultrasound signals emanating from the tissue in response to the emitted light, and suitable for generating a respective optical and/or electrical signal in response to the ultrasound signals. In some embodiments, an acoustic detector 20 may be an ultrasound transducer employing piezoelectric and/or capacitive elements to generate an electrical signal in response to the ultrasound signals emanating from the tissue of the patient. In other words, the ultrasound transducer converts acoustic energy into electrical signals.

In one embodiment, the acoustic detector 20 may be a low finesse Fabry-Perot interferometer mounted on an optical fiber. In such an embodiment, the incident acoustic response emanating from the probed tissue modulates the thickness of a relatively thin polymer film. Such modulation produces a corresponding intensity modulation of light reflected from the polymer film. Accordingly, the acoustic response is converted to optical information, which is transmitted through the optical fiber to an upstream optical detector (which may be any suitable detector). The use of a polymer film as the acoustic detecting surface may allow a relatively high sensitivity to be achieved, even for films of micrometer or tens of micrometers in thickness. In some embodiments, the thin film may be an approximately 0.25 mm diameter disk of 50 micrometer thickness polyethylene terepthalate with an at least partially optically reflective (e.g., approximately 40% reflective) aluminum coating on one side and a mirror reflective coating on the other (e.g., approximately 100% reflective) that form the mirrors of the interferometer. The optical fiber may be any suitable fiber, such as, but not limited to, an approximately 50 micrometer core silica multimode fiber of numerical aperture 0.1 and an outer diameter of approximately 0.25 mm.

In some embodiments, the PA sensor 14 may include a memory and/or other data encoding component, depicted in FIG. 1 as an encoder 24. The encoder 24 may be, but is not limited to, a solid state memory, a resistor, a combination of resistors and/or memory components that may be read or decoded by the monitor 16 (such as, but not limited to, via a reader/decoder 26) to provide the monitor 16 with information about the PA sensor 14, and/or the like. For example, the encoder 24 may encode information about the PA sensor 14 and/or the components thereof (such as, but not limited to, information about the light source 18 and/or the acoustic detector 20). Such encoded information may include, but is not limited to, information about the configuration and/or location of the PA sensor 14, information about the type of lights source(s) 18 present on the PA sensor 14, information about the wavelengths, pulse frequencies, pulse durations, and/or pulse energies which the light source(s) 18 are capable of emitting, information about the nature of the acoustic detector 20, and/or the like. Such information may allow the monitor 16 to select appropriate algorithms and/or calibration coefficients for calculating the patient's physiological parameters.

The PA sensor 14 may be operatively attached (e.g., communicatively coupled) to the monitor 16 via any suitable connection, such as, but not limited to, using a cable (not shown), using one or more wires (not shown), using a wireless communication link, and/or the like. In one embodiment, signals from the acoustic detector 20 (and decoded data from the encoder 24, if present) may be transmitted to the monitor 16. The monitor 16 may include data processing circuitry (such as, but not limited to, one or more processors 28, one or more application specific integrated circuits (ASICS; not shown), and/or the like) coupled to an internal bus 30. Also connected to the bus 30 may be a RAM memory 32, a speaker 34 and/or a display 36. The display 36 and/or the speaker 34 may be used to convey information about the calculated physiological parameters to a user. In some embodiments, a time processing unit (TPU) 38 may provide timing control signals to a light drive circuitry 40, which may control operation of the light source 18. For example, control of the operation of the light source may include, but is not limited to, activation and/or deactivation of the light source 18, the duration of activation of the light source 18 (i.e., how long the light source 18 is operated during a particular cycle), how frequently the light source 18 is activated, whether multiple light sources 18 are used, the multiplexed timing for different light sources 18, and/or the like.

In addition or alternatively to the light drive circuitry 40, the TPU 38 may control and/or contribute to operation of the acoustic detector 20 such that timing information for data acquired using the acoustic detector 20 may be obtained. Such timing information may be used in interpreting the acoustic response data and/or in generating physiological parameters of interest from such acoustic response data. For example, the timing of the ultrasound signal data acquired using the acoustic detector 20 may be associated with the light emission profile of the light source 18 during data acquisition. Likewise, in some embodiments, data acquisition by the acoustic detector 20 may be gated (e.g., via a switching circuit 42) to account for differing aspects of light emission. For example, operation of the switching circuit 42 may allow for separate (i.e., discrete) acquisition of data that corresponds to different respective wavelengths of light emitted at different times.

In some embodiments, the received signal from the acoustic detector 20 may be amplified (e.g., via amplifier 44), may be filtered (e.g., via a filter 46), and/or may be digitized if initially analog (e.g., via an analog-to-digital converter 48). The digital data may be provided directly to the processor 28, may be stored in the RAM 32, and/or may be stored in a queued serial module (QSM) 50 prior to being downloaded to the RAM 32 as the QSM 50 fills up. In some embodiments, there may be separate, parallel paths for separate amplifiers, filters, and/or A/D converters provided for different respective light wavelengths and/or spectra used to generate the acoustic response data.

The data processing circuitry (e.g., the processor 28) may derive one or more physiological parameters based on data generated by the PA sensor 14. For example, based at least in part upon data received from the acoustic detector 20, the processor 28 may calculate the physiological parameters using various algorithms. In some embodiments, such algorithms may use coefficients, which may or may not be empirically determined, that relate the detected acoustic response generated in response to pulses of light at a particular wavelength and/or wavelengths to a given concentration and/or quantity of a constituent of interest. In some embodiments, the data processing circuitry (e.g., the processor 28) may communicate with the TPU 38 and/or the light drive circuitry 40 to spatially modulate the wave front of light emitted by the light source 18, for example based on one or more algorithms.

In some embodiments, processor 28 may access and/or execute coded instructions from one or more storage components of the monitor 16, such as, but not limited to, the RAM 32, a ROM 52, and/or a mass storage device 54. For example, code encoding executable algorithms may be stored in the ROM 52 and/or the mass storage device 54 and accessed and/or operated according to instructions for the processor 28. Such algorithms, when executed and provided with data from the PA sensor 14, may calculate a physiological parameter of the patient. Once calculated, the physiological parameter(s) may be displayed on the display 36 for a user to monitor and/or review. Examples of the mass storage device 54 include, but are not limited to, a magnetic and/or solid state hard drive and/or memory, an optical disk and/or memory, and/or the like.

FIG. 2 is a schematic illustration of an exemplary embodiment of the sensor fixture 12. The sensor fixture 12 is configured to be affixed to the patient's skin at various locations. The sensor fixture 12 is configured to hold the PA sensor 14 (shown in FIGS. 1 and 5) such that the PA sensor 14 is operatively attached with the patient's skin for measuring one or more various physiological parameters of the patient.

The sensor fixture 12 includes a bracket 56 and an acoustic coupling agent 58. As will be described below, the acoustic coupling agent is held by the bracket 56. The acoustic coupling agent 58 is configured to allow the transmission of both acoustic energy and light therethrough. Specifically, the acoustic coupling agent 58 is configured to allow light emitted from the light source 18 (shown in FIGS. 1 and 5) of the PA sensor 14 to be transmitted through the acoustic coupling agent 58 to the patient. The acoustic coupling agent 58 is configured to allow the acoustic response of the patient to be transmitted through the acoustic coupling agent 58 to acoustic detector 20 (shown in FIGS. 1 and 5) of the PA sensor 14. In other words, the acoustic coupling agent 58 transfers acoustic energy from the skin of the patient to the acoustic detector 20. The acoustic coupling agent 58 may be any type of coupling agent that is configured to allow the transmission of both acoustic energy and light therethrough, such as, but not limited to, a gel media, a cream, a fluid, a paste, an ointment, an ultrasound gel, and/or the like.

In some embodiments, the sensor fixture 12 includes a sponge 60 that is held by the bracket 56, as will be described below. The sponge 60 is impregnated with the acoustic coupling agent 58. The sponge 60 provides a matrix for the acoustic coupling agent 58. In some alternative embodiments, the sensor fixture 12 does not include the sponge 60, and the acoustic coupling agent 58 is held within the bracket 56 without using a matrix or using a different matrix besides a sponge. For example, in some alternative embodiments, the acoustic coupling agent 58 is held within the bracket 56 without using a matrix and a user presses on the bracket 56 (e.g., compresses the bracket 56 between the patient's skin and the PA sensor 14) to push the acoustic coupling agent 58 out of the bracket 56. Moreover, in other alternative embodiments, the bracket 56 is designed as a blister pack that will burst to enable the acoustic coupling agent 58 to egress onto the patient's skin and/or the PA sensor 14. In still other alternative embodiments, the acoustic coupling agent 58 is not held within the bracket 56, but rather is manually applied to the patient's skin.

The sponge 60 is configured to allow the transmission of both acoustic energy and light therethrough. Specifically, the sponge 60 is configured such that the sponge 60 facilitates and/or does not interfere with the transmission of light through the acoustic coupling agent 58 to the patient; and the sponge 60 is configured such that the sponge 60 facilitates and/or does not interfere with transmission of the acoustic response through the acoustic coupling agent 58 to acoustic detector 20. The sponge 60 may be fabricated from any material(s), and may have any structure, configuration, arrangement, and/or the like that enables the sponge 60 to allow the transmission of both acoustic energy and light therethrough.

The bracket 56 is configured to be affixed to the patient's skin at various locations, as will be described below. The bracket 56 includes a body 62 having a cavity 64, a patient side 66, and a sensor side 68. The patient side 66 of the bracket 56 faces the patient's skin when the bracket 56, and thus the sensor fixture 12, is affixed to the patient's skin. The patient side 66 of the bracket 56 may or may not engage the patient's skin when the sensor fixture 12 is affixed to the patient's skin. For example, an adhesive may extend between the patient's skin and the patient side 66 when the sensor fixture 12 is affixed to the patient's skin. In the exemplary embodiments shown herein, the patient side 66 is opposite the sensor side 68. But, in other embodiments, the patient side 66 may extend at a non-parallel angle relative to the sensor side 68.

The acoustic coupling agent 58 and the sponge 60 (when included) are held within the cavity 64 of the bracket 56. The patient side 66 of the bracket 56 includes an opening 70 that extends through the patient side 66 into the cavity 64. The opening 70 exposes the acoustic coupling agent 58 along the patient side 66 of the bracket 56 when the sensor fixture 12 is affixed to the patient's skin. The opening 70 enables the acoustic coupling agent 58 to engage the patient's skin when the sensor fixture 12 is affixed to the patient's skin. The opening 70 may be referred to herein as a “patient opening”. The sponge 60 may be held within the cavity 64 of the bracket 56 using any means, such as, but not limited to, using tension (i.e., compressed between portions of the bracket 56), using pins, and/or the like. In some embodiments, the sponge 60 simply rests within the cavity 64 of the bracket 56, whether or not the sponge 60 can float within the cavity 64.

The bracket 56 is configured to hold the PA sensor 14 such that the sensor side 68 of bracket 56 faces the PA sensor 14. The sensor side 68 of the bracket 56 includes an opening 72 that extends through the sensor side 68 into the cavity 64. The opening 72 exposes the acoustic coupling agent 58 along the sensor side 68 of the bracket 56 when the PA sensor 14 is held by the sensor fixture 12. The opening 72 enables the acoustic coupling agent 58 to engage the acoustic detector 20 of the PA sensor 14 when the PA sensor 14 is held by the sensor fixture 12. The opening 72 may be referred to herein as a “sensor opening”.

The sensor fixture 12 may include one or more cover sheets 74 and/or 76 for sealing the openings 70 and/or 72, respectively. For example, the cover sheet 76 may be configured to extend along the patient side 66 of the bracket 56 such that the cover sheet 76 covers, and thereby closes, the opening 70. Such a cover sheet 76 may facilitate containing the acoustic coupling agent 58 and/or the sponge 60 within the cavity 64 of the bracket 56 when the sensor fixture 12 is not in use (e.g., when the sensor fixture 12 is not affixed to the patient's skin). The cover sheet 74 may be configured to extend along the sensor side 68 of the bracket 56 such that the cover sheet 74 covers, and thereby closes, the opening 72. Such a cover sheet 74 may facilitate containing the acoustic coupling agent 58 and/or the sponge 60 within the cavity 64 of the bracket 56 when the sensor fixture 12 is not in use (e.g., when the PA sensor 14 is not held by the sensor fixture 12). The cover sheets 74 and 76 are shown as exploded relative to the bracket 56 in FIG. 2.

The sensor side 68 of the bracket 56 includes a sensor cradle 78 that is configured to hold the PA sensor 14. The sensor cradle 78 holds the PA sensor 14 such that when the sensor fixture 12 is affixed to the patient's skin, the light source 18 of the PA sensor 14 is configured to emit light into the tissue and/or blood at the localized region of the patient being interrogated (e.g., a blood vessel). Moreover, when the PA sensor 14 is held by the sensor cradle 78, the acoustic detector 20 of the PA sensor 14 is operatively attached to the acoustic coupling agent 58 for receiving acoustic energy therefrom. Accordingly, when the sensor fixture 12 is affixed to the patient's skin and the PA sensor 14 is held by the sensor cradle 78, the acoustic detector 20 of the PA sensor 14 is operatively attached to coupling agent 58 for receiving the acoustic response from the patient through the acoustic coupling agent 58.

As shown in FIG. 2, the sensor cradle 78 includes a sensor reception area 80, which is defined along the sensor side 68 of the bracket 56 between two opposing mechanical connector elements 82 of the sensor cradle 78. The sensor reception area 80 is configured to receive the PA sensor 14. The mechanical connector elements 82 are configured to secure the PA sensor 14 to the bracket 56 within the sensor reception area 80 such that the PA sensor 14 is held by the sensor fixture 12.

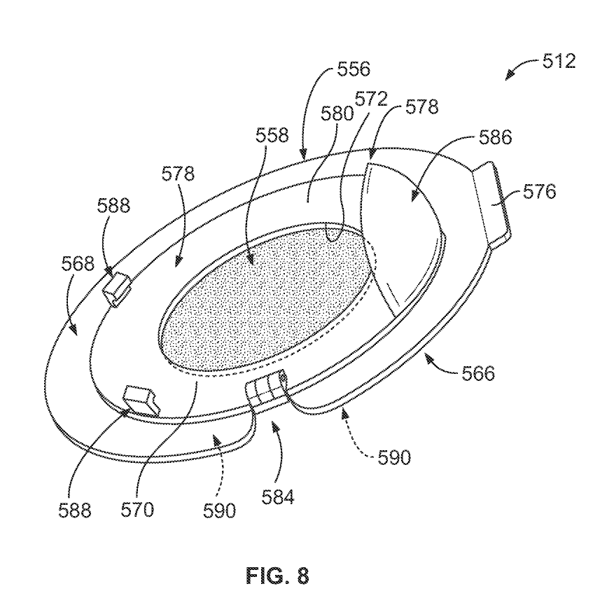

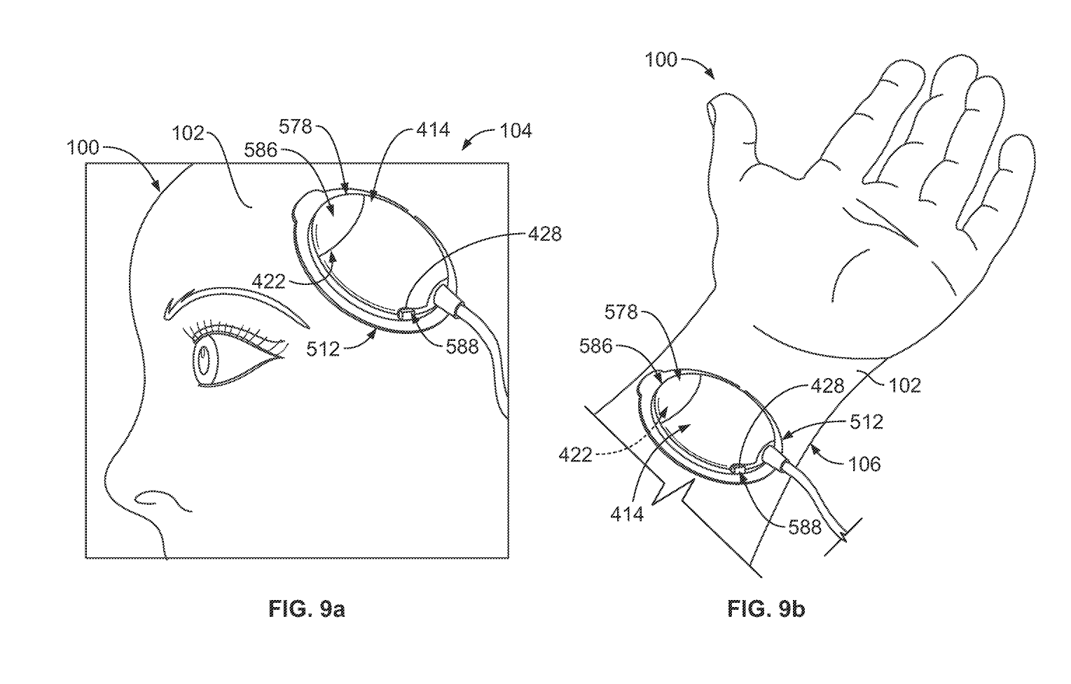

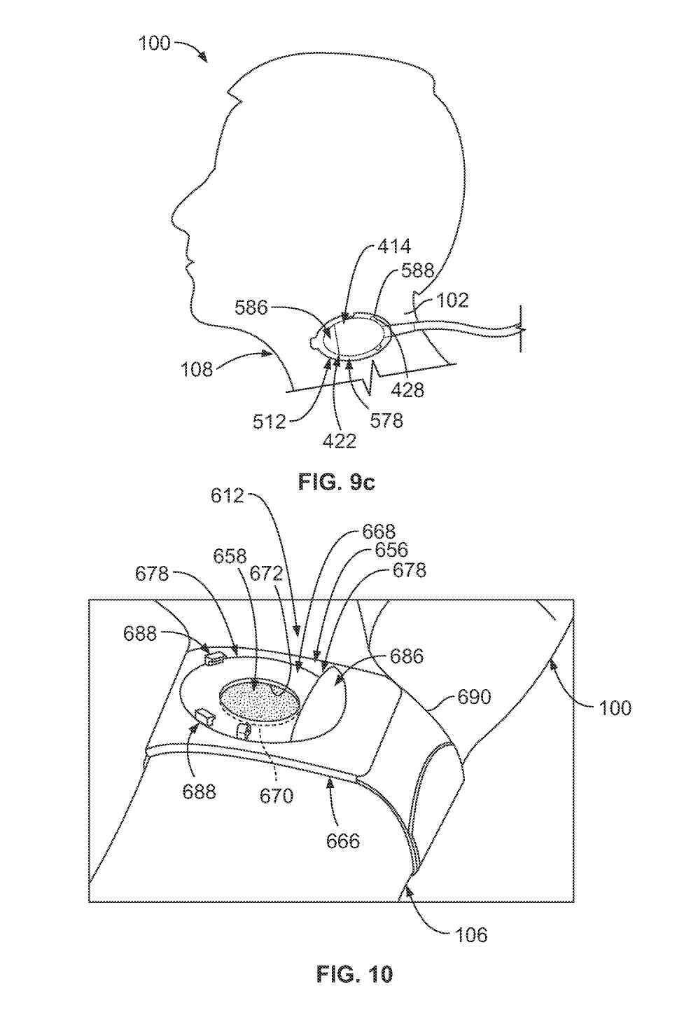

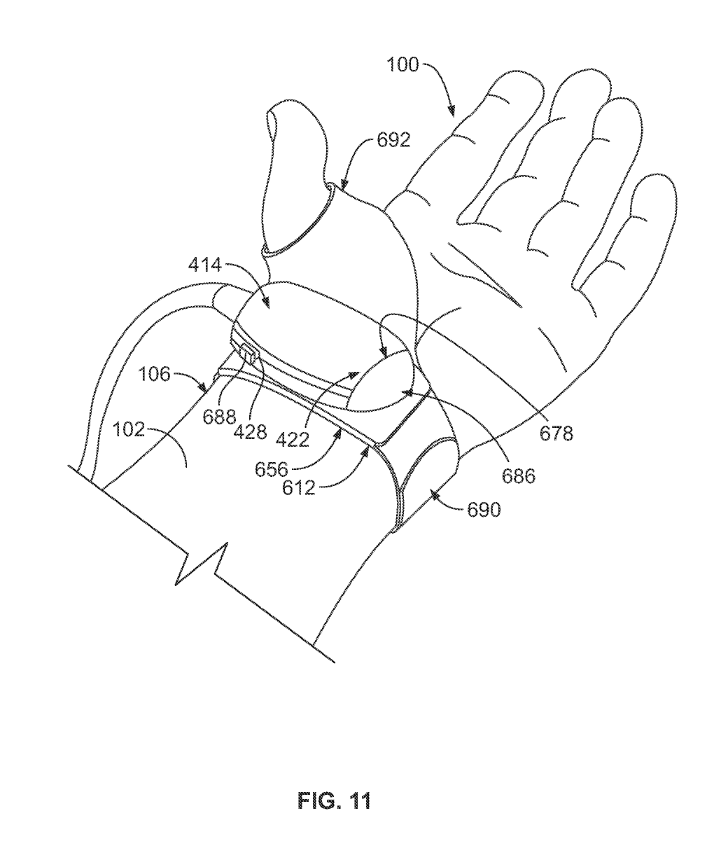

The sensor cradle 78 may include any other components in addition or alternative to the sensor reception area 80 and/or any mechanical connector elements 82. The sensor cradle 78 may include any number of the mechanical elements 82, which may have any relative arrangement (e.g., to at least partially define a sensor reception area 80 having any size and/or shape). The sensor cradle 78 may be configured to hold the PA sensor 14 using any suitable mechanical connection structure, such as, but not limited to, using a snap-fit connection, a press-fit connection, a slide tension (i.e., interference) connection, a threaded fastener, a latch, a lock, and/or the like. For example, each of any mechanical connector elements 82 of the sensor cradle 78 may have any suitable mechanical connection structure. In one specific embodiment, the sensor cradle 78 of the bracket 56 includes a guide element (e.g., the guide element 586 shown in FIGS. 8 and 9, and the guide element 686 shown in FIGS. 10 and 11), which is configured to engage the PA sensor 14 therein for orienting the PA sensor 14 relative to the bracket 56. In such an embodiment, the sensor cradle 78 may also include a mechanical connector element (e.g., the mechanical connector element 588 shown in FIGS. 8 and 9, and the mechanical connector element 688 shown in FIGS. 10 and 11) that is configured to hold the PA sensor 14 in engagement with the guide element.

It should be understood that the sensor cradle 78 is shown only in general schematic form in FIG. 2. Accordingly, the sensor cradle 78 is not limited to the size and shape shown in FIG. 2, nor is the sensor cradle 78 limited to the mechanical connection structure shown in FIG. 2 that is used to hold the PA sensor 14. Rather, the size, the shape, and/or the mechanical connection structure of the sensor cradle 78 may be configured to hold a PA sensor 14: that has any particular size, that has any particular shape, that has any particular arrangement of the light source 18 and/or the acoustic detector 20, that is configured to measure any particular physiological parameter(s), and/or the like. The size, shape, configuration, and/or the like of the sensor cradle 78 and/or the various components thereof may depend on: the location(s) on the patient's body where the PA sensor 14 is configured to measure the physiological parameter(s); the particular size, shape, configuration, and/or the like of the PA sensor 14 that is to be held by the sensor cradle 78; the amount of light and/or acoustic energy that is to be transmitted through the sensor fixture 12; the particular physiological parameter(s) being measured; and/or the like. For example, the sensor cradle 78 and/or one or more components thereof (e.g., the sensor reception area 80) may have a size and shape that is complementary with the size and shape of a particular PA sensor 14. One example of providing a component of the sensor cradle 78 with a size and shape that is complementary with a particular PA sensor 14 includes providing the segment of the sensor side 68 that defines a bottom of the sensor reception area 80 with a curvature that is complementary with the curvature of a particular PA sensor 14. Moreover, in some embodiments, the bracket 56 is at least partially flexible for complying with the shape of the PA sensor 14. Such a complementary curvature and/or flexible manner may facilitate a better fit between the bracket 56 and the PA sensor 14, which may enable the PA sensor 14 to more accurately measure physiological parameters of the patient.

The bracket 56 may be configured to be affixed to the patient's skin using any suitable affixing structure, such as, but not limited to, using an adhesive, using suction, using a wrist band, using a neck band, using an ankle band, using an arm band, using an ear clip, and/or the like. Any type of adhesive may be used. In some embodiments, the adhesive is an adhesive that is specifically designed to adhere to human skin. In embodiments wherein the bracket 56 is affixed to the patient's skin at least partially using an adhesive, in addition or alternative to sealing the opening 70, the cover sheet 76 may be used to cover, and thereby protect, a portion or all of the adhesive when the sensor fixture 12 is not in use (e.g., before and/or after the sensor fixture 12 is affixed to the patient's skin).

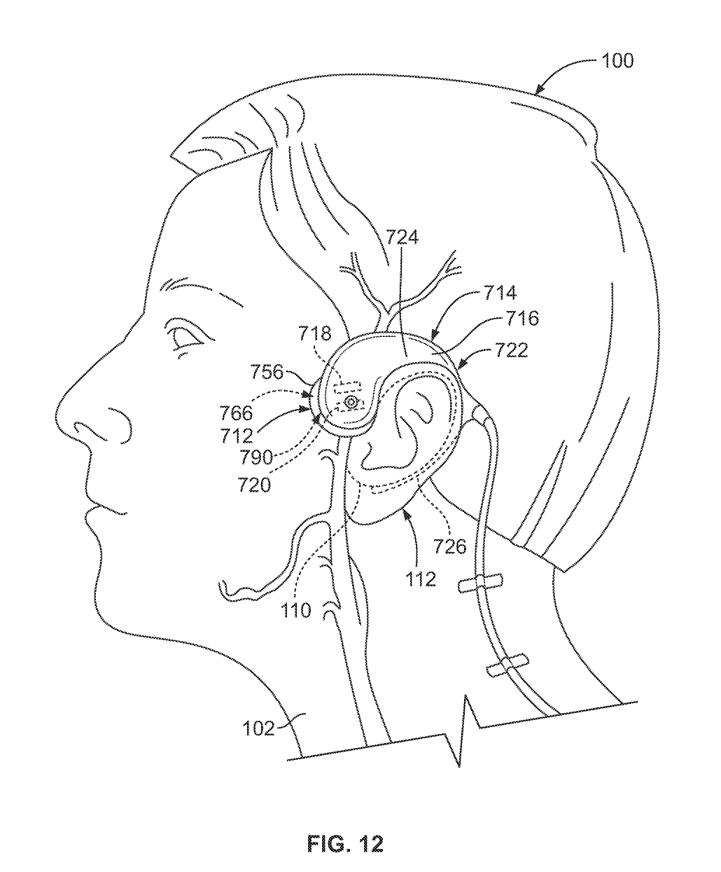

In one specific embodiment of affixing the bracket 56 to the patient's skin, the patient side 66 of the bracket 56 includes an adhesive (not shown in FIG. 2; e.g., the adhesive 590 shown in FIGS. 8 and 9, and the adhesive 790 shown in FIG. 12) extending on at least a portion of the patient side 66, such that the bracket 56 can be affixed to the patient's skin by adhering the adhesive to the patient's skin. Specifically, in such an embodiment, the adhesive forms a mechanical and/or chemical connection with the patient side 66 of the bracket 56 and with the patient's skin, such that the adhesive mechanically connects the patient side 66 of the bracket 56 to the patient's skin. In such an embodiment wherein an adhesive mechanically connects the patient side 66 of the bracket 56 to the patient's skin, a portion of the patient side 66 may or may not engage the patient's skin.

In another specific embodiment of affixing the bracket 56 to the patient's skin, the sensor fixture 12 includes a band (e.g., the wrist band 690 shown in FIGS. 10 and 11) that is configured to be received around the patient (e.g., around a wrist, arm, ankle, and/or neck of the patient). In such an embodiment, the bracket 56 of the sensor fixture 12 is mounted to the wrist band, and the PA sensor 14 is held by the bracket 56. When the wrist band is received around the patient's wrist, the PA sensor 14 is operatively attached to the patient's skin such that the PA sensor 14 is configured to emit light into the tissue and/or blood at the localized region of the patient being interrogated (e.g., a blood vessel), and such that the PA sensor 14 is configured to receive the acoustic response from the patient through the acoustic coupling agent 58.

FIG. 2 is merely intended to represent a general schematic form of the sensor fixture 12 and the various components thereof. The sensor fixture 12 and the various components thereof are not limited to the size, shape, configuration, and/or the like shown in FIG. 2. Moreover, as shown in FIG. 2, the sensor fixture 12 and the various components thereof are not configured for use with any particular PA sensor 14. Rather, the sensor fixture 12 and each of the various components thereof may have any other size, shape, configuration, and/or the like than is shown in FIG. 2. For example, the sensor fixture 12 and the various components thereof may be configured for use with a PA sensor: that has any particular size; that has any particular shape; that has any particular arrangement of the light source 18 and/or the acoustic detector 20; that is configured to measure any particular physiological parameter(s); and/or the like. Moreover, and for example, although the bracket 56 is shown in FIG. 2 as having the shape of a parallelepiped and each of the openings 70 and 72 is shown in FIG. 2 as having a rectangular shape, each of the bracket 56, the opening 70, and the opening 72 may have any other shape.

The size, shape, configuration, and/or the like of the sensor fixture 12 and/or the various components thereof (e.g., bracket body 62, the opening 70, the opening 72, the sensor cradle 78, the acoustic coupling agent 58, the sponge 60) may depend on: the location(s) on the patient's body where the PA sensor 14 is configured to measure the physiological parameter(s); the particular size and/or shape of the PA sensor 14 that is to be held by the sensor fixture 12; the amount of light and/or acoustic energy that is to be transmitted through the sensor fixture 12; the particular physiological parameter(s) being measured; and/or the like. For example, the patient side 66 of the bracket 56 may have a curvature that is complementary with the curvature of the patient's skin at the location(s) on the patient's body where the physiological parameter(s) are to be measured. Moreover, in some embodiments, the bracket 56 is at least partially flexible for complying with the shape of one or more locations on the patient's body. Such a complementary curvature and/or flexible manner may facilitate a better fit between the bracket 56 and the patient's body, which may enable the PA sensor 14 to more accurately measure physiological parameters of the patient.

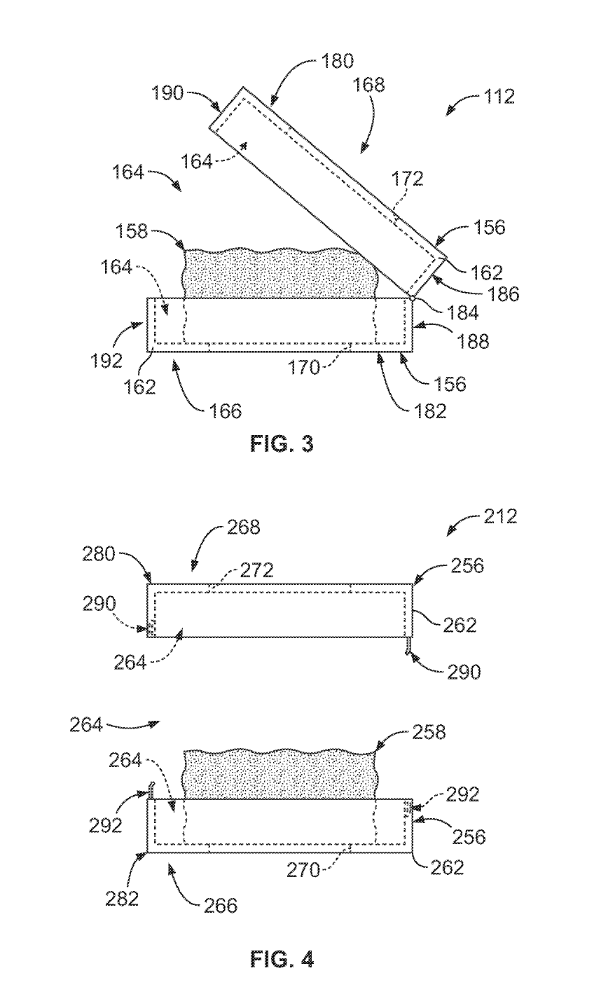

In the general schematic of FIG. 2, the body 62 of the bracket 56 is shown as a single unitary body. But, the bracket body 62 may have any number of components. For example, in some embodiments, the body 62 of the bracket 56 includes two or more shells (e.g., the shells 180 and 182 shown in FIG. 3 and the shells 280 and 282 shown in FIG. 4) that are connected together using any suitable type of mechanical connection, such as, but not limited to, using at least one of a hinge, a living hinge, a clam shell arrangement, a snap-fit connection, a press-fit connection, a slide tension (i.e., interference) connection, a threaded fastener, a latch, a lock, and/or the like. Fabricating the bracket body 62 using two or more shells may ease the positioning of the acoustic coupling agent 58 and/or the sponge 60 within the cavity 64 of the bracket 56. For example, instead of inserting the acoustic coupling agent 58 and/or the sponge 60 into the cavity 64 through the opening 70 and/or 72, two or more shells may be at least partially separated to enable the acoustic coupling agent 58 and/or the sponge 60 to be positioned within the cavity 64.

For example, FIG. 3 is a schematic illustration of another exemplary embodiment of a sensor fixture 112. The sensor fixture 112 includes a bracket 156 and an acoustic coupling agent 158 held by the bracket 156. The bracket 156 includes a body 162 that is defined by at least two shells 180 and 182. A cavity 164 of the bracket 156 is defined between the shells 180 and 182. The shell 180 includes a sensor side 168 of the bracket 156, which includes an opening 172. The shell 182 includes a patient side 166 of the bracket 156, which includes an opening 170. The shells 180 and 182 may each be referred to herein as an “upper shell” and/or a “lower shell”. The opening 172 may be referred to herein as a “sensor opening”, while the opening 170 may be referred to herein as a “patient opening”.

As should be apparent from FIG. 3, the shells 180 and 182 are discrete components from each other. The shells 180 and 182 are mechanically connected together at a hinge 184. Specifically, ends 186 and 188 of the shells 180 and 182, respectively, are connected together by the hinge 184. The shells 180 and 182 and hinge 184 define a clamshell arrangement wherein the shells 180 and 182 are rotatable relative to each other about the hinge 184. The shells 180 and 182 are rotatable about the hinge 184 between an open position and a closed position. In the closed position, respective ends 190 and 192 of the shells 180 and 182 are engaged such that the cavity 164 is only accessible through the openings 170 and 172. For example, the body 162 is substantially similar to the body 62 (shown in FIG. 2) of the bracket 56 (shown in FIGS. 2 and 5) when the shells 180 and 182 are in the closed position. In the open position, the ends 190 and 192 of the shells 180 and 182, respectively, are spaced apart from each other such that the cavity 164 is accessible between the shells 180 and 182. The shells 180 and 182 are shown in the open position in FIG. 3.

When the shells 180 and 182 are in the closed position, the ends 190 and 192 may be held together using any suitable mechanical connection structure (not shown), such as, but not limited to, using a snap-fit connection, a press-fit connection, a slide tension (i.e., interference) connection, a threaded fastener, a latch, a lock, and/or the like. The hinge 184 may be any suitable type of hinge, such as, but not limited to, a discrete hinge that is mounted to both of the ends 186 and 188 of the respective shells 180 and 182, a living hinge, and/or the like.

FIG. 4 is a schematic illustration of another exemplary embodiment of a sensor fixture 212. FIG. 4 illustrates an embodiment wherein a bracket 256 of the sensor fixture 212 includes at least two shells 280 and 282 that are connected together by a snap-fit connection. The sensor fixture 212 includes the bracket 256 and an acoustic coupling agent 258 held by the bracket 256. The bracket 256 includes a body 262 that is defined by the shells 280 and 282. A cavity 264 of the bracket 256 is defined between the shells 280 and 282. The shell 280 includes a sensor side 268 of the bracket 256. The shell 282 includes a patient side 266 of the bracket 256. The sides 268 and 266 of the bracket 256 includes respective openings 272 and 270. The shells 280 and 282 may each be referred to herein as an “upper shell” and/or a “lower shell”. The opening 272 may be referred to herein as a “sensor opening”, while the opening 270 may be referred to herein as a “patient opening”.

The shells 280 and 282 are discrete components from each other that can be mechanically connected together using a snap-fit connection. Specifically, each of the shells 280 and 282 includes one or more snap-fit connectors 290 and 292, respectively. The snap-fit connector(s) 290 of the shell 280 cooperate with corresponding snap-fit connectors 292 of the shell 282 to mechanically connect the shells 180 together. The snap-fit connectors 290 and 292 can be disengaged to enable the shells 280 and 282 to be relatively positioned in an open position. In the open position, the shells 180 and 282 are spaced apart from each other such that the cavity 264 is accessible between the shells 280 and 282. The shells 280 and 282 are shown in the open position in FIG. 3.

In the closed position, the shells 280 and 282 are engaged with each other such that the cavity 264 is only accessible through the openings 270 and 272. For example, the body 262 is substantially similar to the body 62 (shown in FIG. 2) of the bracket 56 (shown in FIGS. 2 and 5) when the shells 280 and 282 are in the closed position.

Each of the snap-fit connectors 290 and each of the snap-fit connectors 292 may be any type of snap-fit connector having any suitable structure. Moreover, although two are shown, each shell 280 and 282 may include any number of the respective snap-fit connectors 290 and 292.

FIG. 5 is a schematic illustration of the PA sensor system 10 illustrating the PA sensor 14 operatively attached to a patient 100. The sensor fixture 12 is affixed to the surface of skin 102 of the patient 100 at a measurement site. In some embodiments, a cover sheet (e.g., the cover sheet 76 shown in FIG. 2) is removed to expose the acoustic coupling agent 58 along the patient side 66 of the bracket 56. As the sensor fixture 12 is affixed to the patient's skin 102, the acoustic coupling agent 58 egresses through the opening 70 and covers the patient's skin 102 at the measurement site. The acoustic coupling agent 58 thereby operatively attaches to the patient's skin 102 for receiving the acoustic response of the patient 100 therefrom. In some embodiments, the sponge 60 is compressed (e.g., by pressing the bracket 56 against the patient's skin and/or by compressing the sponge 60 between the patient's skin and the PA sensor 14) to egress the acoustic coupling agent 58 through the opening 70.

The measurement site at which the sensor fixture 12 is affixed to the patient's skin 102 may be any location on the patient's body suitable for measuring any physiological parameter(s) of the patient 100 (e.g., any of the physiological parameters described above relating to blood flow, cardiac output, and/or blood oxygen saturation). Such locations include, but are not limited to, a location that is adjacent (e.g., extends over) a blood vessel of the patient 100, a location that is adjacent an artery (e.g., the superficial temporal artery, the maxillary artery, the common carotid artery, and the radial artery) of the patient 100, a location that is adjacent an organ and/or region of interest of the patient 100, a location that is adjacent an ear (e.g., the ear 112 shown in FIGS. 12 and 13) of the patient 100, and/or the like. As used herein, the term “adjacent” a blood vessel and the term “adjacent” an artery is intended to mean that the location extends over the blood vessel or the artery such that the blood vessel or artery passed under the location.

The PA sensor 14 is installed in the sensor cradle 78 of the sensor fixture 12 such that the sensor cradle 78, and thus the sensor fixture 12, holds the PA sensor 14. In some embodiments, a cover sheet (e.g., the cover sheet 74 shown in FIG. 2) is removed to expose the acoustic coupling agent 58 along the sensor side 66 of the bracket 56. The PA sensor 14 is held by the sensor fixture 12 such that the acoustic detector 20 and the light emitter 18 of the PA sensor 14 are each aligned with the opening 72 of the bracket 56. As the PA sensor 14 is installed to the sensor fixture 12, the acoustic coupling agent 58 may egress through the opening 72 and into contact with the acoustic detector 20 and/or the light emitter 18. In some embodiments, the sponge 60 is compressed (e.g., by pressing the PA sensor 14 against the bracket 56 and/or by compressing the sponge 60 between the patient's skin and the PA sensor 14) to egress the acoustic coupling agent 58 through the opening 72. When the PA sensor 14 is held by the sensor fixture 12 as shown in FIG. 5, the acoustic detector 20 is operatively attached to the acoustic coupling agent 58 for receiving the acoustic response of the patient 100 therefrom through the opening 72. Moreover, the light emitter 18 is configured to emit light through the opening 72 and through the acoustic coupling agent 58 (and the sponge 60, if included).

In some embodiments, the sensor fixture 12 is configured to be affixed, and the sensor cradle 78 is configured to hold the PA sensor 14, such that the acoustic detector 20 of the PA sensor 14 is oriented approximately perpendicular to a blood vessel of the patient 100. Such an approximately perpendicular orientation may facilitate measurement of a parameter that relates to blood flow and/or cardiac output of the patient 100. Moreover, in embodiments wherein the patient side 66 of the bracket 56 extends at a non-parallel angle relative to the sensor side 68, the sensor fixture 12 may include one or more reflectors (not shown) for directing light and acoustic energy between the sides 66 and 68.

When the PA sensor 14 is held by the sensor fixture 12 as shown in FIG. 5, the PA sensor 14 is operatively attached with the patient's skin 102 for measuring the desired physiological parameter(s) of the patient 100. Specifically, the light emitter 18 emits light through the opening 72, through the acoustic coupling agent 58 (and the sponge 60, if included), through the opening 70, through the patient's skin 102, and into the tissue and/or blood of interest. The light emitted into the tissue and/or blood generates an acoustic response, which is transmitted from the tissue and/or blood to the acoustic coupling agent 58 through the patient's skin 102. The acoustic coupling agent 58 transmits the acoustic response to the acoustic detector 20 of the PA sensor 14.

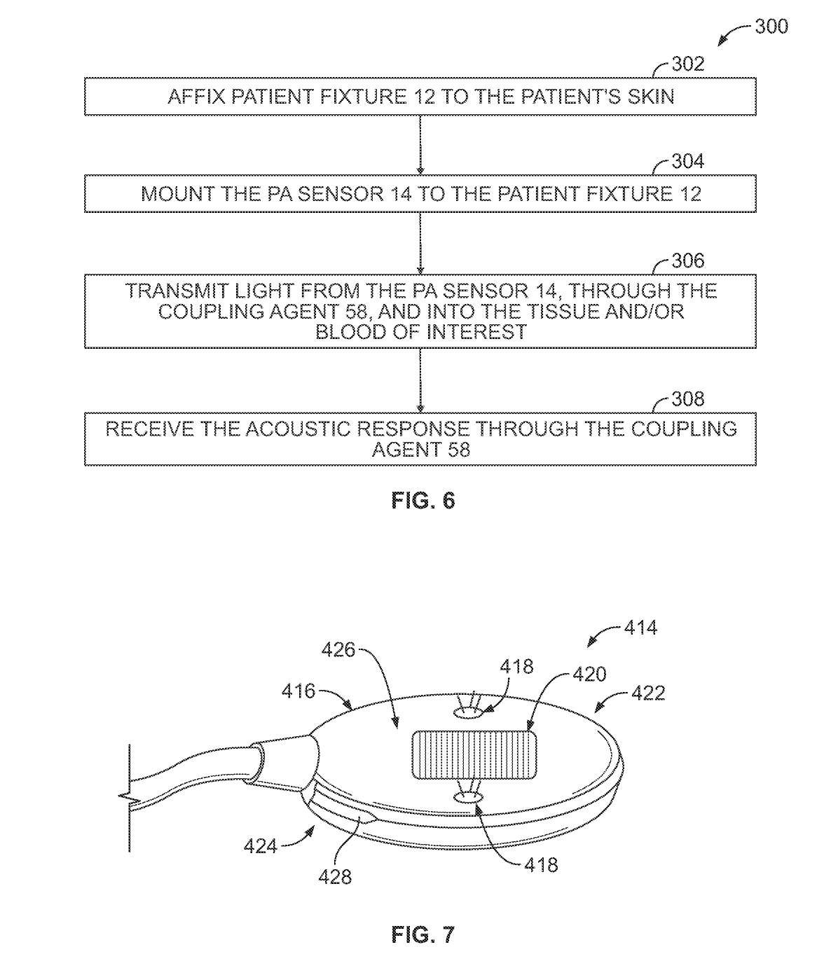

FIG. 6 is a flowchart illustrating an exemplary embodiment of a method 300 for measuring one or more physiological parameters of a patient (e.g., the patient 100 shown in FIG. 5) using the PA sensor system 10 (shown in FIGS. 1 and 5). The method 300 includes, at 302, affixing the sensor fixture 12 (shown in FIGS. 1, 2, and 5) to skin (e.g., the skin 102 shown in FIG. 5) of the patient adjacent tissue and/or blood of interest (e.g., an artery of the patient) such that the acoustic coupling agent 58 (shown in FIGS. 2 and 5) of the sensor fixture 12 engages the patient's skin.

At 304, the method 300 includes mounting the PA sensor 14 to the sensor fixture 12. The PA sensor 14 is mounted to the sensor fixture 12 by installing the PA sensor 14 to the sensor cradle 78 (shown in FIGS. 2 and 5) of the sensor fixture 12. The affixing and mounting steps 302 and 304, respectively, are performed such that the PA sensor 14 is operatively attached with the patient's skin for transmitting light through the sensor fixture 12 and into the tissue and/or blood of interest, and for receiving an acoustic response from the patient through the acoustic coupling agent 58 (shown in FIGS. 2 and 5). The affixing step 302 may be performed before the mounting step 304, however, the method 300 is not limited to performing the affixing step 302 before the mounting step 304. Rather, the mounting step 304 may be performed before the affixing step 302, or the mounting step 304 and the affixing step 302 may be performed simultaneously.

At 306, the method 300 includes transmitting light from the PA sensor 14, through the acoustic coupling agent 58, and into the tissue and/or blood of interest. The light transmitted to the tissue and/or blood of interest generates an acoustic response. At 308, the method 300 includes receiving, at the acoustic detector 20 (shown in FIGS. 2 and 5) the acoustic response from the tissue and/or blood of interest through the acoustic coupling agent 58. In response to receiving the acoustic response, the PA sensor 14 may generate one or more signals that represent the physiological parameter(s) that are desired to be measured. In addition or alternatively, the signals generated by the PA sensor 14 in response to the detected acoustic response represent one or more base physiological parameters that are used by the monitor 16 (shown in FIGS. 1 and 5) to calculate the physiological parameter(s) that are desired to be measured. In some embodiments, the PA sensor 14 includes a visual and/or audio indicator that indicates that there is a reliable operative attachment between the PA sensor 14 and the patient for measuring one or more physiological parameters of the patient with relative accuracy.

Referring again to FIG. 2, the sensor fixture 12 may be sold or supplied to healthcare providers, and/or an intermediate party, as part of the PA sensor system 10 (shown in FIG. 1), whether supplied or sold as attached to a PA sensor 14 (shown in FIGS. 1 and 5). Alternatively, the sensor fixture 12 may be sold or supplied to healthcare providers, and/or an intermediate party, as an individual component. The sensor fixture 12 may be supplied, sold, shipped, and/or stored in a hermetically sealed package, for example, to facilitate preventing damage to, to facilitate preventing contamination of, to facilitate preventing degradation of, and/or to facilitate maintaining a sterilization of any portion of the sensor fixture 12. In some embodiments, a portion(s) or an entirety of the sensor fixture 12 may be sterilized and/or disinfected prior to packaging.

Optionally, the sensor fixture 12 is disposable in that the sensor fixture 12 is intended for a single use only. As used herein, the terms “disposable” and “single use” are intended to mean that a disposable, single use, sensor fixture 12 is used for one and only one patient, and thereafter discarded. For example, a disposable, single use, sensor fixture 12 may be used for one and only one measurement procedure on one and only one patient, and thereafter discarded. Alternatively, a disposable, single use, sensor fixture 12 may be used for a plurality of measurement procedures on one and only one patient, and thereafter discarded. When used for a plurality of measurement procedures on one patient, the disposable, single use, sensor fixture 12 is only applied to the patient one and only one time. However, the sensor fixture 12 may be repositioned on the one and only one patient, for example, to accommodate different measurement locations for different measurements and/or to obtain more accurate measurements. The PA sensor 14 that is held by a disposable, single use, sensor fixture 12 may be discarded along with the disposable, single use, sensor fixture 12 after the measurement procedure(s). Alternatively, the PA sensor 14 can be reused with a different sensor fixture 12 and/or with a different patient 100.

The material(s), size, shape, thickness(es), and/or any other properties, attributes, and/or the like of the sensor fixture 12 may be selected to facilitate providing and/or configuring the sensor fixture 12 as disposable and single use. In embodiments wherein the sensor fixture 12 is a disposable, single use, sensor fixture, the sensor fixture 12 may include a temper element that indicates whether the bracket 56 has been opened (e.g., the shells 80 and 82 have been opened to expose the cavity 64, the cover sheets 74 and/or 76 have been removed, and/or the like). In embodiments wherein the sensor fixture 12 is not a disposable, single use, sensor fixture, a user may replace the sponge 60, the acoustic coupling agent 58, the cover sheets 74 and/or 76, and/or any adhesive between uses of the sensor fixture 12.

FIG. 7 is a perspective view of one specific exemplary embodiment of a PA sensor 414. The PA sensor 414 may be used with the PA sensor system 10 (shown in FIGS. 1 and 5). The PA sensor 414 illustrates one example of a PA sensor that is configured to be held by the specific sensor fixture embodiments described below (e.g., the sensor fixture 512 shown in FIGS. 8 and 9, and the sensor fixture 612 shown in FIGS. 10 and 11). The PA sensor 414 includes a housing 416. The housing 416 extends from a front segment 422 to a rear segment 424. The housing 416 holds one or more light sources 418 and one or more acoustic detectors 420. The light sources 418 and the acoustic detectors 420 are exposed along a patient side 426 of the PA sensor 414.

The housing 416 of the PA sensor 414 includes one or more mechanical connector elements 428 that are configured to cooperated with one or more corresponding mechanical connector elements (e.g., the mechanical connector element 588 shown in FIGS. 8 and 9, and the mechanical connector element 688 shown in FIGS. 10 and 11) of a corresponding sensor fixture to hold the PA sensor 414 to the corresponding sensor fixture. Each mechanical connector element 428 may have any suitable mechanical connection structure, such as, but not limited to, a snap-fit structure, a press-fit structure, a slide tension (i.e., interference) structure, a threaded fastener, a latch structure, a lock structure, and/or the like. In the exemplary embodiment of the PA sensor 414, each mechanical connector element 428 is a snap-recess that is configured to receive a snap-fit connection element therein. In the exemplary embodiment of the PA sensor 414, the PA sensor 414 includes two opposite mechanical connector elements 428 (only one is visible herein), but, the PA sensor 414 may include any number of the mechanical connector elements 428.

FIG. 8 is a perspective view of one specific exemplary embodiment of a sensor fixture 512. The sensor fixture 512 may be used with the PA sensor system 10 (shown in FIGS. 1 and 5). Moreover, the sensor fixture 512 illustrates one example of a sensor fixture that is configured to hold the PA sensor 414 (shown in FIGS. 7, 9, and 11). The sensor fixture 512 includes a bracket 556 that includes a patient side 566 and a sensor side 568. An acoustic coupling agent 558 is held by the bracket 556 and is exposed along the sides 566 and 568 through openings 570 and 572 of the sides 566 and 568, respectively. In the exemplary embodiment, the bracket 556 includes two shells 580 and 582 that are connected together at a hinge 584. The opening 572 may be referred to herein as a “sensor opening”, while the opening 570 may be referred to herein as a “patient opening”.

The sensor side 568 of the bracket 556 includes a sensor cradle 578 that is configured to hold the PA sensor 414. Optionally, the sensor cradle 578 includes a guide element 586 that is configured to engage the PA sensor 414 to orient the PA sensor 414 relative to the bracket 556. In the exemplary embodiment of the sensor fixture 512, the guide element 586 is a hood that is configured to receive the front segment 422 (shown in FIGS. 7, 9, and 11) of the PA sensor 414. The sensor cradle 578 also includes one or more of the mechanical connector elements 588, which are configured to cooperate with the mechanical connector elements 428 of the PA sensor 414 to hold the front segment 422 within the guide element 586. Each mechanical connector element 588 may have any suitable mechanical connection structure, such as, but not limited to, a snap-fit structure, a press-fit structure, a slide tension (i.e., interference) structure, a threaded fastener, a latch structure, a lock structure, and/or the like. In the exemplary embodiment of the sensor fixture 512, each mechanical connector element 588 is a snap-arm that is configured to be received within the snap-recess of the mechanical connector 428 with a snap action. Although two are shown, the sensor cradle 578 may include any number of the mechanical connector elements 588. Moreover, in addition or alternative to the hood, the guide element 586 may have any other suitable mechanical guide structure that enables the guide element 586 to engage the PA sensor 414 and orient the PA sensor 414 relative to the bracket 556. Although the exemplary embodiment of the guide element 586 engages the front segment 422 of the PA sensor 414, the guide element 586 may engage the PA sensor 414 at any location(s) thereof. Although only one is shown, the sensor fixture 512 may include any number of guide elements 586.

An adhesive 590 extends on at least a portion of the patient side 566 of the bracket 556. The adhesive 590 is configured to affix the bracket 556, and thus the sensor fixture 512, to the patient's skin at one or more desired measurement locations. The adhesive 590 may extend on any amount and/or locations of the patient side 566 of the bracket 556. Any type of adhesive 590 may be used. In some embodiments, the adhesive 590 is an adhesive that is specifically designed to adhere to human skin. The sensor fixture 512 is shown as including a cover sheet 576 that covers, and thereby protects, a portion or all of the adhesive 590 when the sensor fixture 512 is not in use.

FIG. 9a-9c illustrate the PA sensor 414 held by the sensor fixture 512 and illustrate the sensor fixture 512 affixed to various locations of a patient's body. The PA sensor 414 is held by the sensor cradle 578 of the sensor fixture 512. Specifically, the front segment 422 of the PA sensor 414 is received within the guide element 586. The mechanical connector elements 588 of the sensor cradle 578 are engaged with the corresponding mechanical connector elements 428 of the PA sensor 414 to hold the front segment 422 within the guide element 586, and thus hold the PA sensor 414 to the sensor fixture 512.

The sensor fixture 512 is affixed to the skin 102 of the patient 100 such that the PA sensor 414 is operatively attached to the patient's skin 102. The sensor fixture 512 is shown as affixed to a variety of different locations on the patient's body. For example, FIG. 9a illustrates the sensor fixture 512 affixed to a temple 104 of the patient 100 adjacent the superficial temporal artery of the patient 100. In FIG. 9a, the sensor fixture 512 operatively attaches the PA sensor 414 to the patient's skin 102 for measuring one or more physiological parameters of or associated with the superficial temporal artery.

FIG. 9b illustrates the sensor fixture 512 affixed to a wrist 106 of the patient 100 adjacent a radial artery of the patient 100. In FIG. 9b, the sensor fixture 512 operatively attaches the PA sensor 414 to the patient's skin 102 for measuring one or more physiological parameters of or associated with the radial artery.

FIG. 9c illustrates the sensor fixture 512 affixed to a neck 108 of the patient 100 adjacent a common carotid artery of the patient 100. In FIG. 9b, the sensor fixture 512 operatively attaches the PA sensor 414 to the patient's skin 102 for measuring one or more physiological parameters of or associated with the common carotid artery.

FIG. 10 is a perspective view of another specific exemplary embodiment of a sensor fixture 612. The sensor fixture 612 may be used with the PA sensor system 10 (shown in FIGS. 1 and 5). Moreover, the sensor fixture 612 illustrates another example of a sensor fixture that is configured to hold the PA sensor 414 (shown in FIGS. 7, 9, and 11). The sensor fixture 612 includes a wrist band 690 that is configured to be received around a wrist 106 of a patient 100. The sensor fixture 612 also includes a bracket 656 that is held by the wrist band 690. The bracket 656 includes a patient side 666 and a sensor side 668. An acoustic coupling agent 658 is held by the bracket 656 and is exposed along the sides 666 and 668 through openings 670 and 672 of the sides 666 and 668, respectively. The opening 672 may be referred to herein as a “sensor opening”, while the opening 670 may be referred to herein as a “patient opening”.

The sensor side 668 of the bracket 656 includes a sensor cradle 678 that is configured to hold the PA sensor 414. Optionally, the sensor cradle 678 includes a guide element 686 that is configured to engage the PA sensor 414 to orient the PA sensor 414 relative to the bracket 656. In the exemplary embodiment of the sensor fixture 612, the guide element 686 is a hood that is configured to receive the front segment 422 (shown in FIGS. 7, 9, and 11) of the PA sensor 414 therein. The sensor cradle 678 also includes one or more of the mechanical connector elements 688, which are configured to cooperate with the mechanical connector elements 428 of the PA sensor 414 to hold the front segment 422 within the hood 686. Each mechanical connector element 688 may have any suitable mechanical connection structure, such as, but not limited to, a snap-fit structure, a press-fit structure, a slide tension (i.e., interference) structure, a threaded fastener, a latch structure, a lock structure, and/or the like. In the exemplary embodiment of the sensor fixture 612, each mechanical connector element 688 is a snap-arm that is configured to be received within the snap-recess of the mechanical connector 428 with a snap action. Although two are shown, the sensor cradle 678 may include any number of the mechanical connector elements 688. Moreover, in addition or alternative to the hood, the guide element 686 may have any other suitable mechanical guide structure that enables the guide element 686 to engage the PA sensor 414 and orient the PA sensor 414 relative to the bracket 656. Although the exemplary embodiment of the guide element 686 engages the front segment 422 of the PA sensor 414, the guide element 686 may engage the PA sensor 414 at any location(s) thereof. Although only one is shown, the sensor fixture 612 may include any number of guide elements 686.

The wrist band 690 may be any type of wrist band, such as, but not limited to, an elastic band, a conventional watch band having two or more links, a strap (e.g., a strap fabricated from leather, fabric, plastic, and/or the like), a rope, a string, a belt, and/or the like. The wrist band 690 is configured to affix the bracket 656, and thus the sensor fixture 612, to the patient's skin at a location that is adjacent a radial artery of the patient 100.

FIG. 11 illustrates the PA sensor 414 held by the sensor fixture 612 and illustrates the sensor fixture 512 affixed to a wrist 106 of a patient 100. The PA sensor 414 is held by the sensor cradle 678 of the sensor fixture 612. The front segment 422 of the PA sensor 414 is received within the guide element 686. The mechanical connector elements 688 of the sensor cradle 678 are engaged with the corresponding mechanical connector elements 428 of the PA sensor 414 to hold the front segment 422 within the guide element 688, and thus hold the PA sensor 414 to the sensor fixture 612.

The sensor fixture 612 is affixed to the skin 102 of the patient 100 such that the PA sensor 414 is operatively attached to the patient's skin 102. The wrist band 690 of the sensor fixture 612 affixes the bracket 656 to the patient's wrist 106 such that the PA sensor 414 is adjacent a radial artery of the patient 100. The sensor fixture 612 thus operatively attaches the PA sensor 414 to the patient's skin 102 for measuring one or more physiological parameters of or associated with the radial artery. The wrist band 690 may include a thumb sling 692, for example to facilitate holding the PA sensor 414 in position over the radial artery.

FIG. 12 is a side elevational view of another specific exemplary embodiment of a PA sensor 714 and another specific exemplary embodiment of a sensor fixture 712. The PA sensor 714 may be used with the PA sensor system 10 (shown in FIGS. 1 and 5). The PA sensor 714 includes a housing 716. The housing 716 holds one or more light sources 718 and one or more acoustic detectors 720.

The housing 716 includes an ear clip 722 that is configured to be received around the base 110 of an ear 112 of a patient 100. The ear clip 722 includes an upper segment 724 that extends over the top of the base 110 of the patient's ear 112. The ear clip 722 includes a lower extension 726 that extends over the bottom of the base 110 of the patient's ear 112. The lower extension 726 may be integrally formed with the remainder of the ear clip 722, or the lower extension 726 may be a discrete component from the remainder of the ear clip 722 that is mechanically connected to the remainder of the ear clip 722.

The PA sensor 714 is held by the sensor fixture 712. The sensor fixture 712 includes a bracket 756 that holds an acoustic coupling agent (not shown). An adhesive 790 extends on at least a portion of a patient side 766 of the bracket 756. The adhesive 790 is configured to affix the bracket 756, and thus the sensor fixture 712, to skin 102 of the patient 100 adjacent a maxillary artery of the patient 100. The adhesive 790 may extend on any amount and/or locations of the patient side 766 of the bracket 756. Any type of adhesive 790 may be used. In some embodiments, the adhesive 790 is an adhesive that is specifically designed to adhere to human skin. In some alternative embodiments, the adhesive 790 is not used, and the ear clip 722 holds the sensor fixture 712 in position over the maxillary artery.

Embodiments of the present disclosure may provide a sensor fixture that operatively attaches a PA sensor to a patient in a relatively quick and simple manner. The sensor fixture may enable the PA sensor to measure various physiological parameters of a patient by probing blood directly in a localized region of interest, such as, but not limited to, in a blood vessel.

Embodiments of the present disclosure may provide a sensor fixture that enables a PA sensor to measure various physiological parameters of a patient in a relatively non-invasive manner. Measurement of the physiological parameters using the sensor fixture may be less invasive than at least some known sensor systems. In some circumstances, situations, and/or the like, it may or may not be possible that the less invasive nature of the measurements provided by the sensor fixture embodiments described and/or illustrated herein cause the patient less discomfort, less injury, less inconvenience, and/or the like.

Embodiments of the present disclosure may provide a disposable, single use, sensor fixture that enables a PA sensor to measure various physiological parameters of a patient. In some circumstances, situations, and/or the like wherein the sensor fixture is a disposable, single use, sensor fixture, the disposable, single use, sensor fixture may facilitate preventing the transmission of infection between patients.

While various spatial and directional terms, such as top, bottom, front, rear, lower, mid, lateral, horizontal, vertical, and/or the like may be used to describe embodiments, it is understood that such terms are merely used with respect to the orientations shown in the drawings. The orientations may be inverted, rotated, or otherwise changed, such that an upper portion is a lower portion, and vice versa, horizontal becomes vertical, and the like.

It is to be understood that the above description is intended to be illustrative, and not restrictive. For example, the above-described embodiments (and/or aspects thereof) may be used in combination with each other. In addition, many modifications may be made to adapt a particular situation or material to the teachings without departing from its scope. While the dimensions, types of materials, and the like described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A sensor fixture for operatively attaching a photoacoustic (PA) sensor to a patient, the sensor fixture comprising:
an acoustic coupling agent configured to allow the transmission of both acoustic energy and light therethrough; and
a bracket configured to be affixed to skin of the patient, the bracket comprising a cavity, a patient side, and a sensor side, the acoustic coupling agent being held within the cavity, the patient side facing the skin of the patient when the bracket is affixed to the skin, the patient side of the bracket comprising a patient opening that is configured to expose the acoustic coupling agent along the patient side, the sensor side of the bracket comprising a sensor opening that is configured to expose the acoustic coupling agent along the sensor side, the sensor side of the bracket comprising a sensor cradle that is configured to hold the PA sensor such that the PA sensor is operatively attached to the acoustic coupling agent for receiving an acoustic response from the patient.

2. The sensor fixture of claim 1, further comprising a sponge that is impregnated with the acoustic coupling agent, the sponge being held within the cavity of the bracket and being configured to allow the transmission of both the acoustic response and light therethrough.

3. The sensor fixture of claim 1, wherein the bracket is configured to be affixed to the skin of the patient at a location that is adjacent an artery of the patient.

4. The sensor fixture of claim 1, wherein the sensor cradle is configured to hold the PA sensor such that an acoustic detector of the PA sensor is oriented approximately perpendicular to an artery of the patient.

5. The sensor fixture of claim 1, wherein the bracket is configured to be affixed to the skin of the patient using at least one of an adhesive and a wrist band.

6. The sensor fixture of claim 1, further comprising a wrist band that is configured to be received around a wrist of the patient, the bracket being mounted to the wrist band such that the wrist band is configured to affix the bracket to the skin of the patient adjacent a radial artery of the patient.

7. The sensor fixture of claim 1, further comprising an adhesive extending on the patient side of the bracket, the adhesive being configured to affix the bracket to the skin of the patient.

8. The sensor fixture of claim 1, wherein the bracket is configured to be affixed to the skin of the patient adjacent an ear of the patient.

9. The sensor fixture of claim 1, wherein the sensor cradle of the bracket is configured to hold the PA sensor using at least one of a snap-fit connection, a press-fit connection, a slide tension connection, a threaded fastener, a latch, or a lock.

10. The sensor fixture of claim 1, wherein the sensor cradle of the bracket comprises a guide element that is configured to engage the PA sensor for orienting the PA sensor relative to the bracket.

11. The sensor fixture of claim 1, wherein the bracket comprises an upper shell and a lower shell, the upper shell comprising the sensor side of the bracket, the lower shell comprising the patient side of the bracket, the upper shell and the lower shell being connected together using at least one of a hinge, a living hinge, a clam shell arrangement, or a snap-fit connection.

12. The sensor fixture of claim 1, wherein the sensor fixture is a disposable, single use, sensor fixture.

13. The sensor fixture of claim 1, further comprising at least one of a cover sheet configured to seal the patient opening when the sensor fixture is not being used or a cover sheet configured to seal the sensor opening when the sensor fixture is not being used.

14. A photoacoustic (PA) sensor system comprising:
a PA sensor having a light source and an acoustic detector, the light source being configured to emit light, the acoustic detector being configured to receive an acoustic response from a patient; and
a sensor fixture for operatively attaching the PA sensor to the patient, the sensor fixture comprising:
an acoustic coupling agent configured to allow the transmission of both the acoustic response and light therethrough; and
a bracket configured to be affixed to skin of the patient, the bracket comprising a cavity, a patient side, and a sensor side, the acoustic coupling agent being held within the cavity, the patient side facing the skin of the patient when the bracket is affixed to the skin, the patient side of the bracket comprising a patient opening that is configured to expose the acoustic coupling agent along the patient side, the sensor side of the bracket comprising a sensor opening that is configured to expose the acoustic coupling agent along the sensor side, the sensor side of the bracket comprising a sensor cradle that is configured to hold the PA sensor such that the acoustic detector of the PA sensor is operatively attached to the acoustic coupling agent for receiving the acoustic response from the patient through the acoustic coupling agent.

15. The system of claim 14, wherein the PA sensor is configured to measure at least one of total hemoglobin (tHb) concentration, oxyhemoglobin saturation (SO₂), or cardiac output (CO).

16. The system of claim 14, wherein the sensor fixture is a disposable, single use, sensor fixture.

17. The system of claim 14, wherein the bracket is configured to be affixed to the skin of the patient using at least one of an adhesive, a wrist band, or an ear clip.

18. The system of claim 14, wherein the sensor cradle of the bracket is configured to hold the PA sensor using at least one of a snap-fit connection, a press-fit connection, a slide tension connection, a threaded fastener, a latch, or a lock.

19. The system of claim 14, wherein the bracket comprises an upper shell and a lower shell, the upper shell comprising the sensor side of the bracket, the lower shell comprising the patient side of the bracket, the upper shell and the lower shell being connected to together using at least one of a hinge, a living hinge, or a snap-fit connection.

20. A method for measuring a physiological parameter of a patient using a photoacoustic (PA) sensor, the method comprising:
affixing a sensor fixture to skin of the patient adjacent an artery of the patient such that an acoustic coupling agent of the sensor fixture engages the skin of the patient;
mounting the PA sensor to the sensor fixture such that an acoustic detector of the PA sensor is operatively attached to the acoustic coupling agent for receiving an acoustic response from the artery of the patient through the acoustic coupling agent;
transmitting light from the PA sensor, through the acoustic coupling agent, and into the artery of the patient to generate the acoustic response; and
at the acoustic detector, receiving the acoustic response from the artery of the patient through the acoustic coupling agent.
